(19) United States
(12) Patent Application Publication
(10) Pub. No.: US 2005/0066404 A1
(43) Pub. Date: Mar. 31, 2005

(54) Protective hand guard

(76) Inventor: Pamela J. Barker, Henderson, NV (US)

Correspondence Address: Pamela J. Barker, 312 Tamarack Dr., Henderson, NV, 89015, US

(21) Appl. No.: 10/806,059

(22) Filed: Mar. 22, 2004

Related U.S. Application Data
(60) Provisional application No. 60/457,222, filed on Mar. 25, 2003.
(60) Provisional application No. 60/484,964, filed on Jul. 3, 2003.

(51) Int. Cl. A47J 45/10; A41D 13/00; A61F 9/00

(52) U.S. Cl. 2/20

(57) ABSTRACT

This protective hand guard utilizes a gelatinous cushion to provide protection and shock absorption for the anatomical structures of the human hand. The hand guard aids in protecting and preserving the hand both externally from cuts and abrasions, and internally from fractures, damage to joints, ligaments, tendons, cartilage, and other anatomical structures found in the human hand. The protection provided by the hand guard is such as to provide a medium for shock absorption, particularly during direct contact or impact sports and or occupations.

### CROSS REFERENCE TO RELATED APPLICATIONS

This claims the benefit of provisional patent # 60457222 filed on Mar. 25, 2003. This also claims the benefit of provisional patent #60484964 filed on Jul. 3, 2003.

### FIELD OF THE INVENTION

The present invention is a device used to aid in protecting the human hand from external and/or internal injury that would commonly occur during impact sports including but not limited to boxing, kickboxing, martial arts, ultimate fighting, or other pugilistic sports. More specifically, the gelatinous cushion acts as a protector and shock absorber to the dorsal and/or palmar surface of the hand, depending on how the hand guard is being worn. In particular, it is the combination of the gelatinous cushion with the specific use of a vinyl or laminate coated fabric with or without stretch, along with the palm strap design that provides a unique and custom-like fit to the individual wearer which conforms to the shape of the hand to provide maximum comfort and performance.

### BACKGROUND OF THE INVENTION

The human hand, by virtue of its attachment to the distal portion of the arm, is a heavily utilized appendage. The hand comes in contact with various surfaces on a daily basis. However, during high impact contact sports and with some occupations, the forces acting on the hand are greatly increased, making the risk of injury greater.

The dorsal surface of the hand is particularly vulnerable to injury because of the lack of natural cushioning around the metacarpalphalangeal joints (knuckles) and the exposure of these joints to direct impact, especially with a clenched fist posture, whether tightly or loosely clenched. A clenched fist is commonly used in contact sports such as boxing and martial arts, where an impact with a person or object is part of the sport. This high speed, direct impact contact exerts tremendous forces over a relatively small surface area. The knuckles lack adequate natural cushioning to help distribute the forces generated, therefore they are vulnerable to external injuries such as abrasions and cuts, as well as internal injuries such as fractures, joint bleeding, bruising, sprains, strains, and other damage to bones, muscles, ligaments, tendons, cartilage, and various anatomical structures. The damage is not limited to the knuckle area. The forces can be distributed from the site of impact down the hand and into the wrist and arm structures as well. It is not uncommon to see boxers with wrist fractures.

The traditional way that boxers and martial artists help to prevent injury to the hand and wrist is through wrapping with specialized tape, muslin, and/or gauze strips. The purpose of this wrapping technique is primarily to support and protect the hand to some degree, and to stabilize the wrist so as to limit range of motion. The materials typically used for hand wrapping, particularly during a sanctioned competition, do not innately provide for much cushioning or shock absorbing ability.

It is standard practice for boxers to wrap their hands for both training and during competition as allowed by the individual state athletic commissions. It is also common for boxers to injure their hands during training and/or during a competition. This compromises their ability to punch with good technique and adequate power when they need it most, i.e. during a boxing match. If a boxer has injured their hand during training or in the early rounds of a fight, they will be unable to fully demonstrate their actual boxing ability, and a fight may be stopped early or lost because of this.

Current hand wrapping techniques include making a small pad out of gauze and placing it over the knuckle area for added protection. The hand is then wrapped with gauze and tape as per boxing regulations. Hand wrapping is highly scrutinized by the individual state athletic commissions so as to limit the possibility of one fighter having a competitive edge over another fighter. Hand wraps are often examined before a fight, with inspectors overseeing the wrapping technique and materials used, and then they are cut off for examination after a fight to ensure compliance with regulations. There has been well publicized controversy over the thickness of the gauze padding and how much has been placed, and some fighters have had to re-wrap their hands to ensure compliance with regulations. In the early 20^{th} century, there were fighters actually accused of adding Plaster of Paris or other hardening agents to firm up their knuckles and give devastating blows to their opponents. Up to this point, the typical product allowed under hand wraps during competition have been a gauze-type product, which has little shock absorbing ability and does little to protect the hand from injury. What is needed is a product that provides more protection and shock absorption to the point of contact, i.e. the knuckle area, to allow the impact forces to better be distributed throughout the hand and therefore better protect the hand from injury. It is important that this protective product be light weight, work together with the traditional hand wrapping techniques and that it fits within a glove yet allows freedom of movement to the wearer.

In examining the products that were commercially available, I found that there was nothing that fully satisfied these requirements. As a professional boxer myself, I wanted something that would protect the instruments of my trade . . . my hands. Through trial and error, I designed the current invention and constructed it myself I made all modifications that were necessary and ultimately ended up with the current invention. I know it works because I have used it myself In reviewing prior art, I found that most previous inventions attempted to control too much. They often used rigid materials such as leather, plastics, and curable resins. They often included thumb attachments which made the appliance too confining and did not allow for adequate freedom of movement. The prior inventions have been too heavy and bulky, and often were not meant to fit within a boxing glove. At the heart of my invention is the use of a gelatinous cushion, which is lightweight and comfortably conforms to the hand. It is thin enough to fit nicely within any standard glove, boxing or martial arts. The use of a gelatinous cushion was purposeful because of the innate ability of gel to distribute forces over a larger surface area, therefore decreasing the point load forces that are generated with direct impact. Because the current invention allows for such tremendous distribution of impact forces, the scope of its use should not be limited to boxing and martial arts. I recognize that many occupations and other sports can benefit from its protective qualities. The design of this protective hand guard allows for it to be worn on either the dorsal or palmar surface of the human hand. The design also allows for it to be housed in a variety of garments and/or equipment pieces, or even stand alone without a housing.

References Sited
U.S. Pat. Documents

| | | |
|---|---|---|
| 5572738 | June, 1995 | Melone |
| 6226795 | April, 2000 | Winningham |
| 5781928 | September, 1996 | Avila |
| 6325747 | October, 1999 | Norblom |
| 5146624 | September, 1989 | Bruckner |
| 4290147 | November, 1979 | Bruckner |
| 6691315 | October, 2002 | Clarke |

### SUMMARY OF THE INVENTION

The protective hand guard is a device that can be worn on the dorsal surface of the hand to provide protection from injury due to the impact forces generated when a fighter punches with a clenched fist. The hand guard is meant to replace or be used in conjunction with the current gauze padding that is traditionally used against the hand and under the hand wraps. The hand guard can be used during training or competition, in conjunction with current and future sanctioned hand wrapping techniques. The design of the protective hand guard is such as to allow it to be reversed and worn on the palm surface of the hand as well as the dorsal surface, depending on the intended use. The placement of the current invention on the hand should not be a limiting factor.

The hand guard consists of a gelatinous cushion, which is anchored to the hand via a palm strap. The size and shape of the palm strap may vary in order to suit specific needs. The gelatinous cushion may come in varying thickness and widths to fit different sized hands and for different applications. Every effort will be made to keep the hand guard as lightweight as possible, therefore different materials may be chosen for different sports, occupations, and/or uses. The hand guard is generally constructed out of a vinyl or laminate coated material, with or without stretch. The hand guard may or may not include finger loop(s).

The hand guard is designed to work in conjunction with conventional wrapping techniques. The hand guard does not have a thumb attachment, allowing the user greater freedom of movement. One purpose of the hand guard is to work in conjunction with or replace the gauze padding that currently is used under a fighter's hand wraps during a competition, thus providing greater protection and shock absorption to the user's hand. Another intent of the hand guard is to provide a mechanism to allow for standardization of cushioning used during a competition so as to avoid delays and controversies that occur with the current use of layered gauze padding. By manufacturing standardized gel hand guards (as per requirements of individual sanctioning bodies) the potential for tampering and therefore creating a “competitive edge” are greatly minimized

At the core of the design is the gelatinous cushion. This cushion acts as a shock absorber and protector to the surface that it covers. The gelatinous consistency is what gives the hand guard its exceptional cushioning and comfort, allowing it to conform to the user's hand for superior comfort. This molding of the gel allows for cushioning between the knuckles that is not possible with traditional gauze padding or with other rigid hand protectors. The gel used can be of various viscosities and may or may not be combined with other materials or ingredients, depending on its intended use. The unique design is the use of gel padding for the indicated purposes of shock absorption and protection. The thickness and consistency may vary without changing the intent of the design. For example, the thickness of the gelatinous cushion may (or may not) be different for a hand guard being used during a professional boxing competition versus a martial arts competition. There is likely to be a difference in size for occupational versus sport use as well. Each use has its own unique needs which will be addressed individually and as allowed by the sanctioning bodies.

Another important component of the hand guard is the specific use of a vinyl or laminate coated material, with or without stretch, in constructing the device. This material is thin, lightweight, yet durable and is water resistant. The user's skin produces moisture when in contact with this material, making it comfortably adhesive to the hand. The specific use of this material provides for a smooth contact surface against the hand rather than the abrasive feel of traditional gauze as it contacts the hand. The combination of this material with the use of gel for the cushion is what allows the hand guard to conform to the shape of the hand. The hand guard feels more like an extension of the person's hand rather than an external device. With the current use of gauze padding during boxing competition and under sanctioned hand wraps, the gauze material greatly absorbs moisture and becomes heavier as the user continues throughout the fight. Traditional gauze can bunch up, dislodge, harden, abrade, flatten out, be tampered with, and/or become heavy. The current invention attempts to solve these problems by providing a standardized gel cushion with a smooth, water resistant surface that may directly contact the skin of the user. Previous inventions have used rigid materials such as leather and plastics to protect the delicate surfaces of the hand. Practically speaking, materials such as leathers, plastics, and curable resins restrict the movement so much as to negatively affect the performance of the user, whether it is a boxer, ultimate fighter, trainer, baseball player, etc. I believe that the current invention solves many of these problems by providing a protective device that is lightweight, uses a thin lightweight fabric which feels comfortable against the skin, does not have a thumb attachment which can be restrictive to the user, and provides superior shock absorption ability through the specific use of a gelatinous cushion which aides in distributing the forces over a larger surface area of the hand, therefore minimizing the point load impact forces that are often generated with punching and other hard impact sports or occupations.

### BRIEF DESCRIPTION OF THE DRAWINGS

All drawings provided are for illustrative purposes only and are not meant to be to scale or as limitations in the design. For example, actual colors, sizes, shapes, fit and/or positioning may vary.

FIG. 1 shows dorsal view of right hand wearing hand protector with 4 finger loop design and palm strap which is not shown in this view. Dorsal surface is protected with gelatinous cushion portion covering knuckle area and to middle of hand. Broken line shows seam dividing gelatinous cushion and cushion stabilizer.

FIG. 2 is palmar view of FIG. 1 showing each finger inserted in finger loops and palm strap across palm.

FIG. 3 shows side view of left hand slightly turned to show partial palmar view with fingers extending upward. Hand protector worn is the same design as that shown in FIGS. 1 and 2.

FIG. 4 shows side view of right hand with fingers extending upward. This is another view of the same design of hand protector as FIGS. 1, 2, and 3.

FIG. 5 shows left hand in a partially clenched fist, another view of the same design as that shown in FIGS. 1, 2, 3, and 4.

FIG. 6 shows dorsal view of right hand wearing another design of the current invention. Knuckle area and just below are protected by the gelatinous cushion, there is no cushion stabilizer. There are no finger loops in this design. The hand protector is held in place by the palm strap.

FIG. 7 shows palmar view of FIG. 6.

FIG. 8 shows side view of left hand slightly turned to show partial palmar view with fingers extending upward. Hand protector worn is the same design as that shown in FIGS. 6 and 7.

FIG. 9 shows side view of right hand with fingers extending upward. This is another view of the same design of the hand protector as FIGS. 6, 7, and 8.

FIG. 10 shows left hand in a partially clenched fist, another view of the same design as that shown in FIGS. 6, 7, 8, and 9.

### DETAILED DESCRIPTION OF THE INVENTION

In any previous and following descriptions, terms such as front, back, top, bottom, reversible, palmar, dorsal, gel, filled, cushion, loops, strap, stretch, padding, gauze, wraps, product, invention, water resistant, user, wearer, fighter, fight, competition, sanctioning, and other like terms are used solely for the purpose of clarity in illustrating the invention and it's novel use, and should not be taken as words of limitation. The drawings are for the purpose of illustrating the invention and are not intended to be exactly to scale or of the exact design parameters to be used in manufacturing the actual product. For example, curves, lines, stitching, thickness, size, and materials may vary depending on the intended use.

The intention of the current invention is to provide superior shock absorption to the hand while still being lightweight and comfortable to the user. It is important that the user not feel restricted in motion by the product. Also, it is important that the product could be used in conjunction with current acceptable hand protection techniques. For instance, the current invention can be used in conjunction with a fighter's hand wrapping regardless of the technique or materials used to wrap the hand. In some cases the current invention can replace the standard gauze pad that is traditionally used under a fighter's hand wraps during training and/or competition, as allowed by the individual state athletic commissions or other sanctioning bodies.

The use of finger loops is optional and may or may not be implemented as appropriate for each use. If the finger loop design is chosen, they slide easily onto the fingers of the user to anchor the hand guard to the surface. Specific size and placement of the finger loops may vary. The gelatinous cushion can be worn over the dorsum of the hand to help protect the knuckles and hand from impact injury. The palm strap can be worn over the palm surface of the hand to secure the gel cushion to the hand. Appropriate hand wraps can be placed directly over the hand guard as directed by the sanctioning bodies. If the current invention is worn in the reversed position, then the gel cushion can be placed on the palm surface of the hand and the palm strap can rest on the dorsal surface of the hand.

The current invention is intended to aid in protecting the hand from injury due to impact forces, frictional forces and blunt trauma that can occur during various sports and occupational applications. Although much in this text is referring to use of the current invention for boxing applications, such description is for illustrative purposes. The scope of the product use should not be limited to any particular sport or occupational application, as it is recognized that it could be useful in many related and/or unrelated sports and occupational venues.

Preferred Embodiment:

At the core of the protective hand guard design is the use of a gelatinous material for its superior shock absorbing qualities, combined with the use of a vinyl or laminate coated fabric which allows the gel cushion to conform to a persons hand while providing for a comfortable, non-restrictive fit. The gel used can be of various viscosities and may or may not be combined with other materials or ingredients. The techniques for constructing a gel based cushion include but are not limited to using gel sheet layers, injected gel, liquid gel filling a chamber, and all other known gel preparations. The preferred embodiment of gel is a gel wax formulation which is melted down then poured into cushion molds to create the appropriate sizing and consistency. Strips of gauze or other such materials can be added to the cushion when it is still hot to provide a more stable and cohesive cushion. The gel may be combined with a variety of materials or ingredients depending on the intended application and should not be limited to a particular preparation. It is recognized that as technology changes, the methods of preparation and materials used may change as well. The essence of the design shall stay the same, particularly the use of a gel substance to form a gelatinous cushion which provides for distribution of forces over a larger surface area, thereby providing protection to the hand when worn. The gel cushion can be encased in a vinyl or laminate coated fabric, which provides a superior fit and comfort. The fabric used may include but not be limited to PVC, PU coated or other vinyl fabric, with or without stretch, including Lycra or spandex backed material. The preferred fabric should have sufficient stretch so as to allow the gel to conform to the user's hand; however stretch is not absolutely required to provide a good fit. The fabric should be lightweight, yet durable enough to withstand repeated impact forces over time.

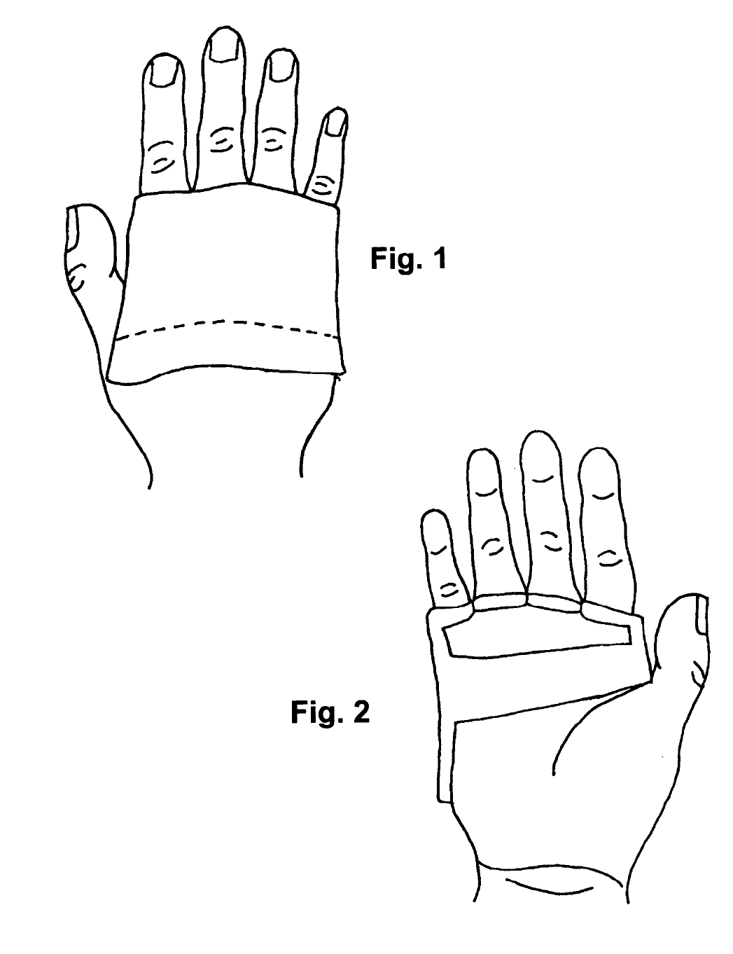

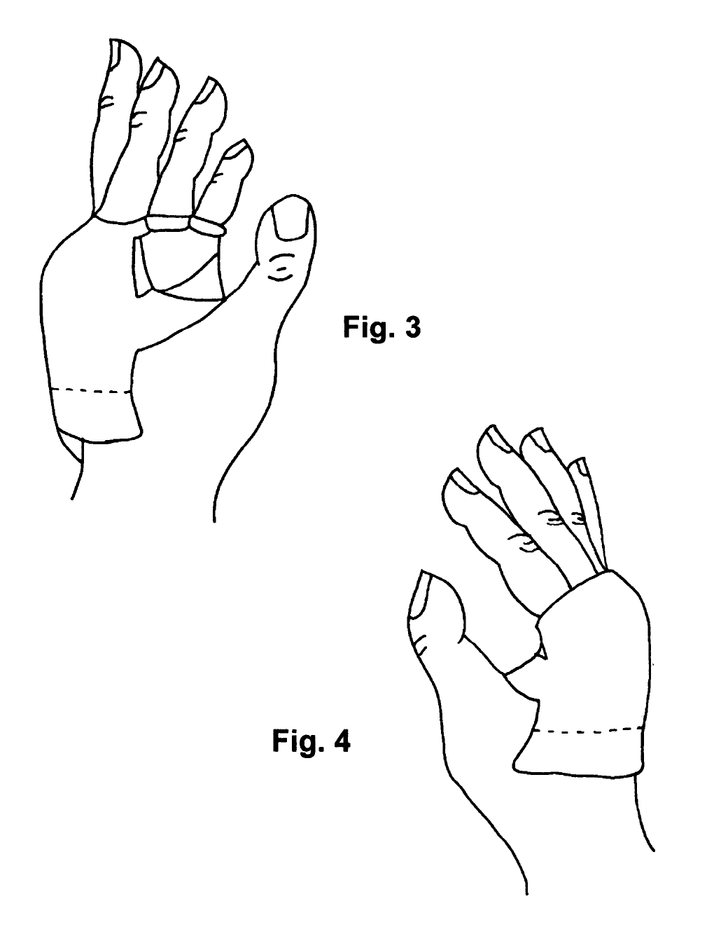

FIGS. 1 and 2 show the dorsal aspect of the human hand, with and without the hand guard. FIG. 1 illustrates the basic competition hand guard design. It should be noted that finger loop(s) can be added as appropriate for certain applications. The finger loop application is not illustrated here. Also, the gel cushion can be extended, or a stabilizing flap added.

The gelatinous cushion is secured to the hand by the palm strap. The palm strap may vary in style, length, width, or size depending on the intended use and preferences.

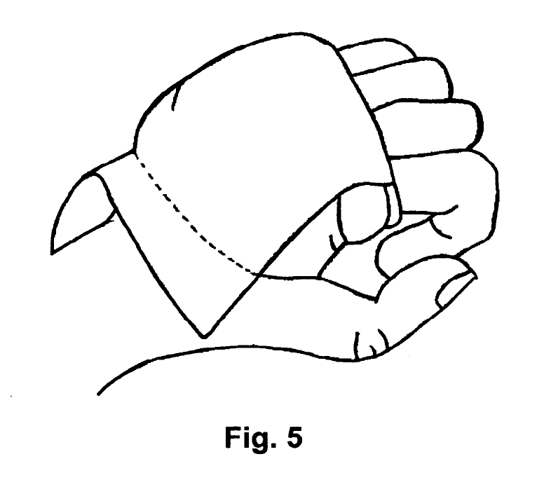

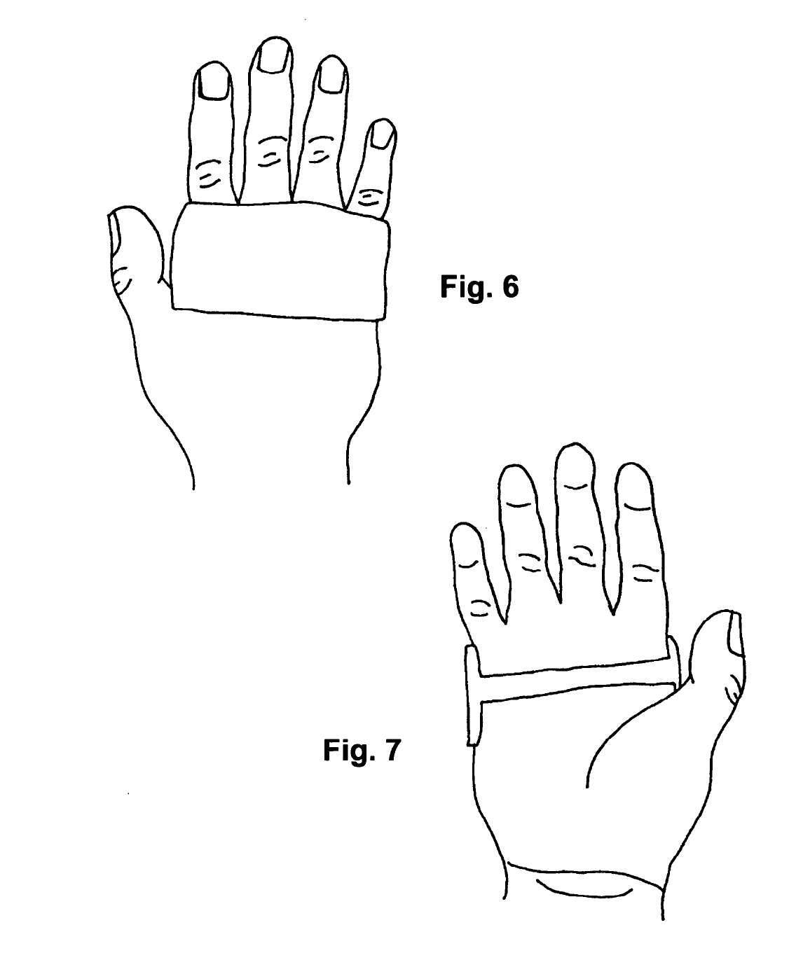

FIGS. 3, 5, and 6 show some of the possible variations in the palm strap design. FIG. 3 illustrates the typical palm strap configuration, which is the preferred embodiment for use in boxing. FIG. 4 shows a palmar view of the hand without the hand guard on it. FIGS. 5 and 6 are examples of 2 possible variations in the palm strap design. Actual needs and use may vary significantly and cannot be limited to the illustrated versions.

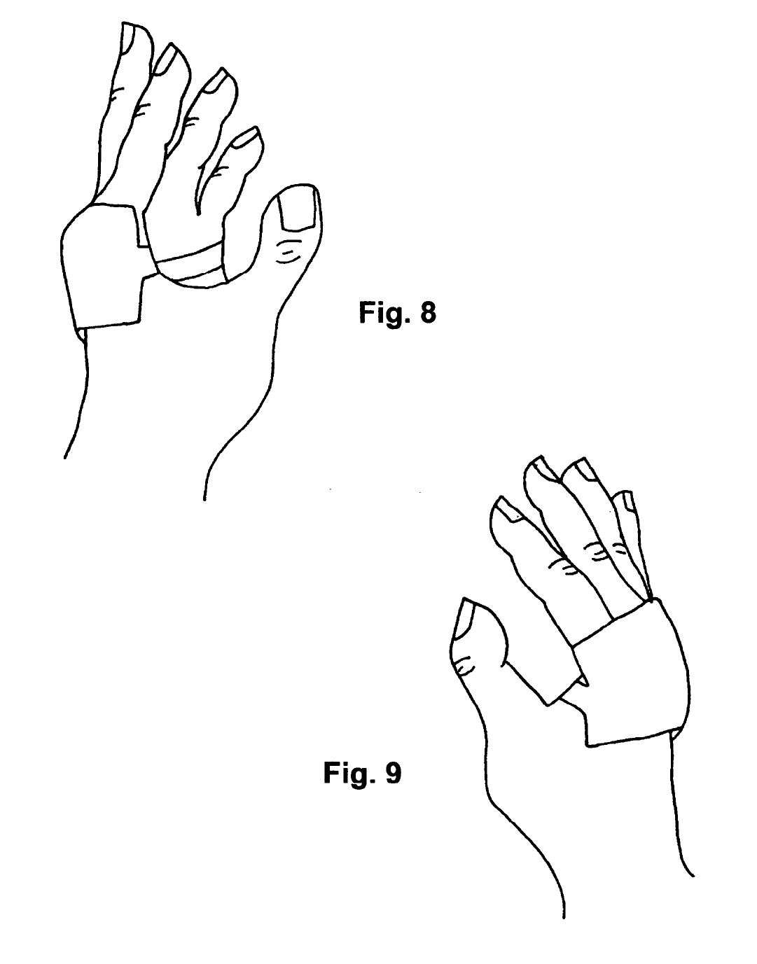

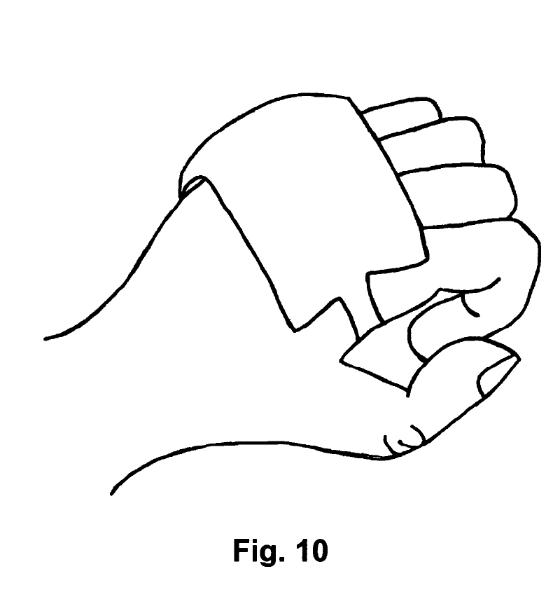

FIGS. 7, 8, 9, and 10 all show a side view of the hand with and without the hand guard on it. These drawings further illustrate the different variations that may be used. The actual protective hand guard may include but not be limited to any of the illustrated versions.

1-3. (canceled)

4. A protective hand guard comprising:
a gelatinous cushion which provides for distribution of forces over a large area, thereby protecting a hand from point load impact forces when said protective hand guard is worn; and
a palm strap to secure the protective hand guard to the hand.

5. The protective hand guard of claim 4, wherein the gelatinous cushion is encased in a vinyl or laminate coated fabric, which allows the protective hand guard to conform to a shape of the hand.

6. The protective hand guard of claim 5, wherein the fabric includes any one of polyvinyl chloride (PVC), polyurethane (PU) coated fabric, Lycra and spandex backed material.

7. The protective hand guard of claim 4, wherein the gelatinous cushion is secured on the protective hand guard such that it protects the dorsal surface of the hand.

8. The protective hand guard of claim 4, wherein the palm strap is arranged on the protective hand guard such that it covers a palm surface of the hand.

9. The protective hand guard of claim 4, wherein the gelatinous cushion is arranged on the protective hand guard such that it protects a palm surface of the hand.

10. The protective hand guard of claim 4, further includes a cushion stabilizer designed to protect a lower region of the dorsal surface of hand.

11. The protective hand guard of claim 4, wherein the gelatinous cushion extends such that it protects a lower region of the dorsal surface of hand.

12. A protective hand guard comprising:
a gelatinous cushion which provides for distribution of forces over a large area, thereby protecting a hand from point load impact forces when worn; and
a finger loop to secure the protective hand guard to the hand.

13. The protective hand guard of claim 12, wherein the gelatinous cushion is encased in a vinyl or laminate coated fabric, which allows the protective hand guard to conform to a shape of the hand.

14. The protective hand guard of claim 13, wherein the fabric includes any one of polyvinyl chloride (PVC), polyurethane (PU) coated fabric, Lycra and spandex backed material.

15. The protective hand guard of claim 12, wherein the gelatinous cushion is secured on the protective hand guard such that it protects the dorsal surface of the hand.

16. The protective hand guard of claim 12, wherein the finger loop is arranged on the protective hand guard such that it goes around a finger of the hand.

17. The protective hand guard of claim 12, wherein the gelatinous cushion is arranged on the protective hand guard such that it protects a palm surface of the hand.

18. The protective hand guard of claim 12, further includes a cushion stabilizer designed to protect a lower region of the dorsal surface of hand.

19. The protective hand guard of claim 12, wherein the gelatinous cushion extends such that it protects a lower region of the dorsal surface of hand.
